(19) United States
(12) Patent Application Publication
(10) Pub. No.: US 2014/0294740 A1
(43) Pub. Date: Oct. 2, 2014

(54) ORAL CARE COMPOSITIONS

(71) Applicants: Myriam Mondin, Seraing (BE); Marie Christine Houben, Alleur (BE); Pierre Lambert, Retine (BE)

(72) Inventors: Myriam Mondin, Seraing (BE); Marie Christine Houben, Alleur (BE); Pierre Lambert, Retine (BE)

(73) Assignee: Colgate-Palmolive Company, New York, NY (US)

(21) Appl. No.: 14/117,069

(22) PCT Filed: Nov. 9, 2011
(86) PCT No.: PCT/US11/59847

§ 371 Date: Jun. 18, 2014

Related U.S. Application Data
(60) Provisional application No. 61/498,056, filed on Jun. 17, 2011.

(51) Int. Cl. A61K 8/25; A61K 8/44; A61K 8/60; A61K 8/46; A61K 8/21; A61Q 11/00

(52) U.S. Cl. CPC A61K 8/25; A61K 8/21; A61Q 11/00; A61K 8/60; A61K 8/463; A61K 8/442
USPC 424/52; 424/49

(57) ABSTRACT

Described herein are compositions comprising a low abrasive silica and a fine silica thickener, and suspended elements, along with methods for making and using the same.

### CROSS-REFERENCE TO RELATED APPLICATIONS

The present application claims priority to U.S. Provisional Application No. 61/498,056, filed Jun. 17 2011, the entirety of which is hereby incorporated by reference.

### BACKGROUND

Gel toothpastes are known and extremely popular with consumers, due m part to their attractive visual aesthetics. In some instances, the gel comprises discrete suspended elements such as film fragments, beads, or capsules, which contribute to the visual attractiveness of the product. However in existing formulations, the gel may be clouded or turbid, obscuring the :suspended elements. Moreover, the suspended elements are a relatively expensive component of the toothpaste, Accordingly, it is desirable to develop toothpaste gels comprising suspended elements having improved gel clarity and reduced cost.

### SUMMARY

In the some embodiments, the present invention provides oral care compositions haying improved clarity and reduced cost (e.g., Composition 1), comprising a low abrasive silica and a fine silica thickener m a ratio of 1.5:1 to 1:1.5, e.g., about 1:1, and suspended elements, e.g., beads, film pieces, or capsules, wherein the composition is substantially free of high cleaning silica, for example Composition 1A, a toothpaste gel composition comprising:

(a) a gel base comprising

i. low abrasive silica, e.g., Zeodent® 114, e.g., 6-10% e.g., about 8%,

ii. fine silica thickener, e.g., Zeodent® 165, e.g., 6-10%, e.g., about 8%,

iii. humectant, e.g., selected from sugar alcohols (e.g., sorbitol), hydrophilic polymers (e.g., polyethylene glycol), polyols (e.g., glycerol), and mixtures thereof, e.g., 40-70%, e.g. about 50%, for example about 48% sorbitol and about 1% polyethylene glycol;

iv. surfactant, e.g., selected from anionic surfactant, e.g., sodium lauryl sulfate; zwitterionic surfactant, e.g., cocamidopropyl betaine; nonionic surfactant, as block copolymer based on ethylene oxide and propylene oxide; and mixtures thereof; e.g., 2-5%, e.g., a combination of about 1.5% sodium lauryl sulfate and about 1.25% cocamidopropyl betaine;

v. an effective amount of a fluoride ion source, e.g., sodium fluoride, about 0.32%,

vi. optionally a structuring agent, e.g., carboxymethyl cellulose, e.g., 0-2%, e.g., about 0.5%,

vii. optionally a colorant, e.g., about 0-0.1%,

viii. optionally an antiplaque agent, e.g. tetrasodium pyrophosphate, e.g., 0-1%, e.g., about 0.5;

ix. optionally a flavoring agent, e.g., sweetener, for example sodium saccharin, cooling flavor, for example, mint flavor and combinations thereof, e.g. 0.5-3%, e.g. about 1.2% mint flavor and about 0.2; and

x. water e.g., 5-20%; and

(b) elements suspended in said gel, e.g., selected from beads, capsules, and film fragments; e.g., in an amount of 0.5-1.5%;

wherein the gel is substantially free of high cleaning silica; and

wherein the gel has a turbidity measurement of less than 200 NTU (nephelometric turbidity units); and

wherein all percentages are with respect to the total weight of the composition.

In some embodiments, the present invention provides a toothpaste gel composition (e.g., Composition 1B) comprising (amounts by weight):

| | |
|---|---|
| low abrasive silica | 7-9% |
| fine silica thickener | 7-9% |
| humectant | 45-55% |
| anionic surfactant | 1-2% |
| zwitterionic surfactant | 1-2% |
| fluoride ion source | 0.1-0.5%, |
| structuring agent | 0-2% |
| colorant | 0-0.1%, |
| antiplaque agents | 0-1% |
| flavoring, | 0.5-3% |
| water | 5-20% |
| suspended elements | 0.5-1.5%. |

Other embodiments provide, for example,
1.1. Composition 1, 1A or 1B wherein the low abrasive silica is amorphous precipitated silica having an oil absorption of greater than about 90 cm³/100 g and an Einlehner hardness of less than 5 mg loss per 100,000 revolutions.
1.2. Any of the foregoing compositions wherein “high cleaning silica” refers to silica having an oil absorption of less than about 90 cm3/100 g and an Einlehner hardness of greater than 5 mg loss per 100,000 revolutions.
1.3. Any of the foregoing compositions wherein the anionic surf surfactant component is selected from the water-soluble salts of alkyl sulfates having about 10 to about 18 carbon atoms in the alkyl radical, and the water-soluble salts of sulfonated esters of fatty acids having about 10 to about 18 carbon atoms., and mixtures thereof;
1.4. Any of the foregoing compositions wherein the anionic surfactant comprises sodium lauryl sulfate;
1.5. Any of the foregoing compositions wherein the amount of anionic surfactant is 0.5-3%, e.g., 1-2%;
1.6. Any of the foregoing compositions wherein the zwitterionic surfactant component comprises a betaine;
1.7. Any of the foregoing, compositions wherein the zwitterionic surfactant component comprises cocamidopropyl betaine;
1.8. Any of the foregoing compositions wherein the amount of zwitterionic surfactant is 1-2%;
1.9 Any of the foregoing compositions wherein the ratio of low abrasive silica to fine silica thickener is about 1:1,
1.10. Any of the foregoing compositions wherein the humectant comprises sorbitol, low molecular weight polyethylene glycol (e.g., having an average molecular weight of 200 to 1000 Daltons, e.g. about 600 Daltons), or mixtures thereof;
1.11. Any of the foregoing compositions wherein the amount of humectant by weight is 45-50%;
1.12. Any of the foregoing compositions wherein the fluoride ion source is sodium fluoride.
1.13 Any of the foregoing compositions comprising an antiplaque-effective amount of tetrasodium pyrophosphate;
1.14. Any of the foregoing compositions comprising an effective amount of a non-cariogenic sweetening agent, e.g., sodium saccharin;
1.15. Any of the foregoing compositions comprising an effective amount of an antibacterial agent, e.g., triclosan;
1.16. Any of the foregoing compositions comprising an anionic polymer, e.g., a copolymer of methyl vinyl ether and maleic anhydride;
1.17 Any of the foregoing compositions comprising an effective amount of an antisensitivity agent, e.g., potassium nitrate;
1.18. Any of the foregoing compositions comprising an effective amount of a basic amino acid in free or salt form, e.g., arginine;
1.19. Any of the foregoing compositions wherein the suspended elements comprise flavor beads;
1.20. Any of the foregoing compositions wherein the refractive index of the gel components is approximately the same;
1.21. Any of the foregoing compositions wherein the gel has a turbidity measurement of 50-150 NTU;
1.22. Any of the foregoing compositions wherein the gel provides a magnifying effect;
1.23. Any of the foregoing compositions comprising the following components by approximate weight percent, all values being rounded to the nearest significant figure, +/−10%:

| | |
|---|---|
| Low abrasive silica: | 8 |
| Fine silica thickener: | 8 |
| Polyethylene glycol 600: | 1 |
| Sorbitol | 50 |
| Sodium lauryl sulfate: | 1.5 |
| Cocamidopropyl betaine: | 1.3 |
| Structuring agent | 0.5 |
| Flavor: | 1.2 |
| Sodium saccharin: | 0.3 |
| Sodium fluoride: | 0.32 |
| Colorant: | 0.03 |
| Suspended elements | 1.1 |
| Water | 30 |

In some embodiments, compositions of the present invention provide an acceptable level of cleaning performance. In some embodiments, compositions of the present invention deliver an acceptable level of cooling sensation. In some embodiments, compositions of the present invention deliver a consumer acceptable level of flavor.

In some embodiments, the present invention provides oral care compositions and methods of using the Sallie that are elective in inhibiting or reducing the accumulation of plaque, reducing levels of acid producing (cariogenic) bacteria, remineralizing teeth, and inhibiting or reducing gingivitis. In some embodiments, the present invention encompasses compositions and methods to clean the oral cavity and provide improved methods of promoting oral health and/or systemic health, including cardiovascular health, e.g., by reducing potential for systemic infection via the oral tissues.

### DETAILED DESCRIPTION

As used throughout, ranges are used as shorthand for describing each and every value that is within the range. Any value within the range can be selected as the terminus of the range.

All references cited herein are hereby incorporated by reference in their entireties.

In the event of a conflict in a definition in the present disclosure and that of a cited reference, the present disclosure controls.

As used herein, the term “structuring agent” refers to a substance which is able to form by itself, or in combination with another substance, a structured network in an aqueous medium, and provide a G′/G″ value of ≧1. wherein G′ is the elastic modulus and G″ is the viscous modulus.

As used herein, “turbidity” refers to the clarity (or lack thereof) in a gel or liquid. Turbidity for purposes of this application is measured using a calibrated nephelometer and is expressed in Nephelometric Turbidity Units (NTU). A lower NTU value indicates a less turbid or clearer gel,

By way of example and not limitation, low abrasive silica includes, for example, synthetic amorphous precipitated silicas having a mean particle size of up to about 20 μm, typically at about 8 to about 14 μm, with an oil absorption structure of greater than about 90 to about 110 cm³/100 g, with an Einlehner hardness of about 2 to about 4 mg loss per 100.000 revolutions, such as ZEODENT® 115 or ZEODENT 114®, marketed by J. M. Huber Corporation, Havre de Grace Md., U. S. A .

In some embodiments, useful silica thickeners include, for example, an amorphous precipitated silica such as ZEODENT® 165 silica. Other (though non-limiting) silica thickeners such as ZEODENT® 163 and/or 167 and ZEOFREE® 153, 177, and/or 265 silicas, all available from J. M. Huber Corporation, Havre de Grace Md., U.S.A, may also be suitable for use in the compositions described herein.

When referred to herein, Einlehner hardness is measured by conventional means. For example, a Brass Einlehner (BE) Abrasion value can be measured through the use of an Einlehner AT-1000 Abrader. In this test, a Fourdrinier brass wire screen is weighed and exposed to the action of a 10% aqueous silica suspension for a fixed number of revolutions, and the amount of abrasion is then determined as milligrams brass lost from the Fourdrinier wire screen per 100,000 revolutions. Disposable supplies required for this test (brass screens, wear plates and PVC tubing) are available from Duncan Associates, Rutland, Vt. and sold as an “Einlehner Test Kit”. Specifically, brass screens (Phosphos Bronze P.M.) were prepared by washing in hot, soapy water (0.5% Alconox) in an ultrasonic bath for 5 minutes, then rinsed in tap water and rinsed again in a beaker containing 150 ml water set in an ultrasonic bath. The screen is rinsed again in tap water, dried in an oven set at 105° C. for 20 Minutes, cooled in a desiccator and weighed. Screens are handled with tweezers to prevent skin oils from contaminating the screens. The Einlehner test cylinder is assembled with a wear plate and weighed screen (red line side down—not abraded side) and clamped in place. The wear plate is used for about 25 tests or until worn badly; the weighed screen is used only once.

As used herein, “oil absorption” is measured using conventional means, e.g., using the ASTA Rub-Out Method D281. For example, oil absorption can be measured using linseed oil by the rubout method. In this test, oil is mixed with a silica and rubbed with a spatula on a smooth surface until a stiff putty-like paste is formed. By measuring the quantity of oil required to have a paste mixture, which will curl when spread out, one can calculate the oil absorption value of the silica—the value which represents the volume of oil required per unit weight of silica to completely saturate the silica sorptive capacity.

In some embodiments, compositions of the present invention comprise a mixture of compatible surfactants. Suitable surfactants are those which are reasonably stable throughout a wide pH range, and may comprise anionic, nonionic and/or zwitterionic surfactants. Examples of suitable surfactants are described more fully, for example, in U.S. Pat. No. 3,959,458, to Agricola et al.; U.S. Pat. No. 3,937,807, to Haefele; and U.S. Pat. No. 4,051,234, to Gieske et al., which are incorporated herein by reference.

In some embodiments, an anionic surfactant is used. In some embodiments, the anionic surfactant component comprises sodium lauryl sulfate. In some embodiments, zwitterionic surfactants may be utilized. In some embodiments, the zwitterionic surfactant component comprises a betaine, e.g., cocamidopropyl betaine. In some embodiments, nonionic surfactants may be suitable for use in the compositions of the present invention. In some embodiments, the nonionic surfactant component comprises a block copolymer based on ethylene oxide and propylene oxide, e.g., a poloxamer. In some embodiments, the mixture of surfactants suitable for use in the compositions of the present invention comprises sodium lauryl sulfate and cocamidopropyl betaine.

In some embodiments, water is present in the oral compositions of the invention. Water, employed in the preparation of commercial oral compositions should be deionized and free of organic impurities. Water amounts as provided herein include the free water which is added to the mixture in preparing the composition, plus that amount which is introduced with other materials such as with sorbitol or any components of the invention.

In some embodiments, it is also desirable to incorporate a humectant to maintain the water levels and prevent the composition from hardening upon exposure to air. Certain humectants can also impart desirable sweetness or flavor to dentifrice compositions. The humectant, on a pure humectant basis, generally includes about 15% to about 70% in some embodiments, or about 30% to about 65% in other embodiments by weight of the dentifrice composition. Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the toothpaste compositions herein. Humectants may also include hydrophilic polymers such as polyethylene glycol, which may also enhance foaming, and contribute to the viscosity of the gel.

In some embodiments, the humectants are non-crystallizing sorbitol and low molecular weight polyethylene glycol (PEG 600).

Levels of active ingredients will vary based on the nature of the delivery system and the particular active. The oral care compositions may include one or more fluoride ion sources, soluble fluoride salts. Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof in certain embodiments the fluoride ion source includes stannous fluoride, sodium fluoride, sodium monofluorophosphate as well as mixtures thereof.

In certain embodiments, the oral care composition of the invention may contain a source of fluoride ions or fluorine-providing ingredient in amounts sufficient to supply about 25 ppm to 25,000 ppm of fluoride ions, generally at least about 500 ppm, e.g., about 500 to about 2000 ppm, about 1000 to about 1600 ppm, e.g., about 1450 ppm. The appropriate level of fluoride will depend on the particular application.

Fluoride ion sources may be added to the compositions of the invention at a level of about 0.01 wt. % to about 10 wt. %.

The compositions of the invention are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided.

The oral care compositions of the invention also may include an agent to increase still further the amount of foam that is produced when the oral cavity is brushed.

Illustrative examples of agents that increase the amount of foam include, but are not limited to polyoxyethylene and certain polymers including, but not limited to, alginate polymers.

The oral care compositions of the invention also may optionally include one or more chelating agents able to complex calcium found in the cell walls of the bacteria. Binding of this calcium weakens the bacterial cell wall and augments bacterial lysis.

Acidic polymers, for example polyacrylate gels, may be provided in the form of their free acids or partially or fully neutralized water soluble alkali metal (e.g., potassium and sodium) or ammonium salts. Certain embodiments include about 1:4 to about 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 119 (MN. 250,000) and S-97 Pharmaceutical Grade (M.W. 70,000), of GAF Chemicals Corporation.

In preparing oral care compositions, it is sometimes necessary to add some thickening material to provide a desirable consistency or to stabilize or enhance the performance of the formulation, and also the structuring agents discussed above may help provide this function as well as the function of enhancing the density and volume of the foam. In certain embodiments, the thickening agents are carboxyvinyl polymers, and carrageenan. Natural gums such as karaya, gum arabic, and gum tragacanth can also be incorporated. Hydroxyethyl cellulose and water soluble salts of cellulose ethers such as sodium carboxymethyl cellulose and sodium carboxymethyl hydroxyethyl cellulose may be used m small amounts, but should be used with care as they may interfere with the enhanced foaming, of the instant invention. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

In addition to the above described components, the embodiments of this invention can contain a variety of optional dentifrice ingredients some of which are described below. Optional ingredients include, for example, but are not limited to, adhesives, sudsing agents, additional flavoring agents, sweetening agents, additional antiplaque agents, additional abrasives, and coloring agents. These and other optional components are further described in U.S. Pat. No. 5,004,597, to Majeti; U.S. Pat. No. 3,959,458 to Agricola et al. and U.S. Pat. No. 3,937,807, to Haefele, all being incorporated herein by reference.

In some embodiments, the compositions of the present invention comprise the following components by approximate weight percent, all values being rounded to the nearest significant figure, +/−10%:

| | |
|---|---|
| Low abrasive silica: | 8 |
| Fine silica thickener: | 8 |
| Polyethylene glycol 600: | 1 |
| Sorbitol | 50 |
| Sodium lauryl sulfate: | 1.5 |
| Cocamidopropyl betaine: | 1.3 |
| Structuring agent | 0.5 |
| Flavor: | 1.2 |
| Sodium saccharin: | 0.3 |
| Sodium fluoride: | 0.32 |
| Colorant: | 0.03 |
| Suspended elements | 1; and |
| Water | 30. |

Some embodiments provide a toothpaste gel composition comprising a. low abrasive silica and a fine silica thickener in a ratio of 1.511 to 111.5; and suspended elements, wherein the composition is substantially free of high cleaning silica and wherein the gel is substantially clear. Some embodiments provide one or more suspended elements.

Some embodiments provide compositions comprising, by weight: a gel base comprising from about 6 to about 10% low abrasive silica; from about 0 to about 10% fine silica thickener; from about 40 to about 70%. of a humectant; from about 2 to about 5% of a surfactant; an effective amount of a fluoride ion source, and from about 5 to about 20% water; and elements suspended in the gel, selected from beads, capsules, and film fragments. In some embodiments, the gel is substantially free of high cleaning silica. In other embodiments, the gel has a turbidity measurement of less than 200 NTU.

Other embodiments provide compositions comprising from about 7 to about 9% low abrasive silica; from about 7 to about 9% fine silica thickener; from about 45 to about 55% humectant; from about 1 to about 2% anionic surfactant; from about 1 to about 2% zwitterionic surfactant; from about 0.1 to about 0.5% of a fluoride ion source; from about 0,1 to about 2% of a structuring agent; an optional colorant; from about 0.1 to about 1% of an antiplaque agent; from about 0.5 to about 3% of a flavoring agent; and from about 0.5 to about 1.5% of a suspended element.

In some embodiments, the low abrasive silica is amorphous precipitated silica having an oil absorption of greater than about 90 cm³/100 g and an Einlehner hardness of less than 5 mg loss per 100,000 revolutions, In some embodiments, high cleaning silica is a silica having an oil absorption of less than about 90 cm³/100 g and an Einlehner hardness of greater than 5 mg loss per 100,000 revolutions.

In some embodiments, the anionic surfactant component is selected from the water-soluble salts of alkyl sulfates having about 10 to about 18 carbon atoms in the alkyl radical, and the water-soluble salts of sulfonated esters of fatty acids having about 10 to about 18 carbon atoms, and mixtures thereof. in some embodiments, the zwitterionic surfactant component comprises a betaine.

In some embodiments, the ratio of low abrasive silica to tine silica thickener is about 1:1.

In other embodiments, the humectant comprises sorbitol, low molecular weight polyethylene glycol (e.g., having an average molecular weight of 200 to 1000 Daltons, e.g. about 600 Daltons), or a mixture thereof.

In some embodiments, the fluoride ion source is sodium fluoride. Some embodiments provide compositions comprising an effective amount of an antisensitivity agent.

In some embodiments, the suspended element is a flavor bead.

In some embodiments, the refractive index of the gel components is approximately the same.

In other embodiments, the composition has a turbidity measurement of less than 150 NTU. In other embodiments, the composition has a turbidity measurement of from about 50 to about 150 NTU. In other embodiments, the composition has a turbidity measurement of from about 75 to about 125. In other embodiments, the composition has a turbidity measurement of from about 90 to about 120. In other embodiments, the composition has a turbidity measurement of from about 95 to about 105.

In some embodiments, the composition surpasses the consumer equity signal. In other embodiments, the compositions demonstrate improved transparency, wherein the improved transparency provides a magnifying effect. In other embodiments, the magnifying effect permits a reduced number of beads to provide the same visual impact. In some embodiments, the improved transparency provides a superior visual impact. In some embodiments, the superior visual impact is provided by the optimal ratio of beads to turbidity.

In some embodiments, the composition maintains an acceptable level of cleaning performance despite a reduction in abrasive concentration. In some embodiments, the composition maintains an acceptable level of cleaning performance despite a significant reduction in abrasive concentration. In some embodiments the composition maintains an acceptable level of cleaning performance despite a greater than 50% reduction in abrasive concentration.

In some embodiments, the present invention involves applying to the oral cavity a safe and effective amount of the compositions described herein.

The Compositions of the invention are additionally useful in methods to reduce harmful bacteria in the oral cavity, for example methods to reduce or inhibit gingivitis, reduce levels of acid producing bacteria, to increase relative levels of arginolytic bacteria, inhibit microbial biofilm formation in the oral cavity, raise and/or maintain plaque pH at levels of at least about pH 5.5 following sugar challenge, reduce plaque accumulation, treat dry mouth, and/or clean the teeth and oral cavity. Enhancing oral health also provides benefits in systemic health, as the oral tissues can be gateways for systemic infections. Good oral health is associated with systemic health, including cardiovascular health.

It is understood that the formulations described herein, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples arc given sold for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### EXAMPLES

### Example 1

Table 1 (below) describes the formulation of a control composition and an exemplary composition of the present invention (“Composition I”).

TABLE 1

| Ingredient | Control | Composition I |
|---|---|---|
| Sorbitol non-crystallizing, 70% solution | 58.57 | 68.00 |
| Water | 8.82 | 8.37 |
| Sodium carboxymethylcellulose | 0.65 | 0.50 |
| Polyethylene glycol 600 | 3.00 | 1.00 |
| Sodium Saccharin | 0.27 | 0.27 |
| Sodium Fluoride | 0.32 | 0.32 |
| Tetra Sodium pyrophosphate | 0.5 | — |
| Colorant | — | 0.0375 |
| High cleaning silica | 10.00 | — |
| Low abrasive silica | 10.00 | 8.00 |
| Silica thickener | 2.75 | 8.00 |
| Flavor | 1.20 | 1.20 |
| Sodium lauryl sulfate | 1.50 | 1.50 |
| Cocamidopropyl betaine | 1.25 | 1.25 |
| Menthol spheres | 1.50 | 1.10 |

### Example 2

Table 2 (below) describes data generated from turbidity measurements of a control composition and Composition I. The turbidity of Composition I and the control composition was measured using a HACH 2100P TUBIDIMETER.

TABLE 2

| | Turbidity of Control (NTU) | Turbidity of Composition I (NTU) |
|---|---|---|
| S1 | 471 | 119 |
| | 441 | 103 |
| | 424 | 102 |
| S2 | 550 | 114 |
| | 503 | 111 |
| | 456 | 119 |
| Mean | 474 | 111 |
| Std Dev | 42 | 7 |

Although the test formulation has nearly a one third reduction in the White Vision Spheres (a relatively expensive component), the greater clarity and magnifying properties of the test gel show off the spheres better, and provide unproved visual aesthetics at lower cost.

Despite the reduction in abrasive amounts, the test formulation provides acceptable cleaning and is highly rated by consumers.

What is claimed is:

1. A toothpaste gel composition comprising
a low abrasive silica and a fine silica thickener in a ratio of 1.5:1 to 1:1.5; and
suspended elements,
wherein the composition is substantially free of high cleaning silica and
wherein the gel is substantially clear.

2. The composition of claim 1 comprising, by weight:
i) gel base comprising
from about 6 to about 10% of a low abrasive silica;
from about 6 to about 10% of a fine silica thickener; and
ii) elements suspended in said gel, selected from beads, capsules, and film fragments;
wherein the gel is substantially free of high cleaning silica; and
wherein the gel has a turbidity measurement of less than 200 NTU.

3. The composition of claim 1 comprising:
from about 7 to about 9% of a low abrasive silica;
from about 7 to about 9% of a fine silica thickener; and
from about 0.5 to about I .5% of suspended elements.

4. The composition of claim 1, wherein the ratio of low abrasive silica to fine silica thickener is about 1:1.

5. The composition of claim 1, wherein the suspended elements comprise flavor beads.

6. The composition of claim 1, wherein the refractive index of the gel components is approximately the same.

7. The composition of claim 1, wherein the gel has a turbidity measurement of from about 50 to about 150 NTU.

8. The composition of claim 1, wherein the composition surpasses the consumer equity signal.

9. The composition of claim 1, wherein the improved transparency provides a magnifying effect.

10. The composition of claim 1, wherein the superior visual impact is provided by the optimal ratio of beads to turbidity.

11. The composition of claim 1, wherein the magnifying effect permits a reduced number of beads to provide the same visual impact.

12. The composition of claim 1, wherein the composition, maintains an acceptable level of cleaning performance despite a reduction in abrasive concentration.

13. The composition of claim 2, wherein the gel base further comprises a humectant, a surfactant and a fluoride source.

14. The composition of claim 13, wherein the humectant is sorbitol and the surfactant is sodium butyl sulfate and cocamidopropyl betaine.

15. The composition of claim 14, wherein the ratio of low abrasive silica to fine silica thickener is about 1:1.
